(19) United States
(12) Patent Application Publication
(10) Pub. No.: US 2007/0032866 A1
(43) Pub. Date: Feb. 8, 2007

(54) Accommodating diffractive intraocular lens

(76) Inventor: Valdemar Portney, Tustin, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/499,934

(22) Filed: Aug. 7, 2006

Related U.S. Application Data
(60) Provisional application No. 60/705,876, filed on Aug. 5, 2005.

(51) Int. Cl. A61F 2/16

(52) U.S. Cl. 623/6.31; 623/6.37

(57) ABSTRACT

One disclosed embodiment of a method includes providing an intraocular lens. The intraocular lens includes a diffractive optical surface having diffractive properties which produce an interference pattern. The method further includes implanting the lens in an eye of a patient such that the diffractive optical surface changes shape in response to action of an ocular structure of the eye. The interference pattern is modified in response to the action of the ocular structure. One disclosed embodiment of an intraocular implant includes a lens body. The lens body comprises a diffractive optical surface having diffractive properties which produce an interference pattern. The lens body is sized and shaped for placement in an anterior portion of a human eye. The lens body is sufficiently flexible to change the shape of the diffractive optical surface in response to ciliary muscle action so that the interference pattern is modified.

### CROSS-REFERENCE TO RELATED APPLICATIONS

This application claims the benefit under 35 U.S.C. § 119(e) of U.S. Provisional Application No. 60/705,876, filed Aug. 5, 2005, titled ACCOMMODATING DIFFRACTIVE INTRAOCULAR LENS, the entire contents of which are hereby incorporated by reference herein and made a part of this specification.

### BACKGROUND

1. Field

Certain embodiments disclosed herein relate to intraocular lenses and, more particularly, to intraocular lenses that permit accommodation.

2. Description of the Related Art

It is a common practice to implant an artificial lens in an eye following such procedures as the removal of a cataract. However, certain currently known artificial lenses suffer from various drawbacks.

### SUMMARY

In certain embodiments, a method comprises providing an intraocular lens. The intraocular lens comprises a diffractive optical surface having diffractive properties which produce an interference pattern. The method further comprises implanting the lens in an eye of a patient such that the diffractive optical surface changes shape in response to action of an ocular structure of the eye. The interference pattern is modified in response to the action of the ocular structure.

In some embodiments, an intraocular implant comprises a lens body. The lens body comprises a diffractive optical surface having diffractive properties which produce an interference pattern. The lens body is sized and shaped for placement in an anterior portion of a human eye. The lens body is sufficiently flexible to change the shape of the diffractive optical surface in response to ciliary muscle action so that the interference pattern is modified. In some embodiments, at least about 80 percent of the optical output of the diffractive optical surface is in a single diffraction order.

In some embodiments, an intraocular implant comprises an optical element sized for insertion into a human eye. The optical element has a diffractive optical surface. The diffractive optical surface has an unaccommodated state in which the diffractive optical surface creates a first interference pattern and an accommodated state in which the diffractive optical surface creates a second interference pattern which differs from the first interference pattern. The optical element is sufficiently flexible to change from the unaccommodated state to the accommodated state in response to ciliary muscle action.

In some embodiments, an intraocular implant comprises an optical element sized for insertion into a human eye. The optical element has a diffractive optical surface. The diffractive optical surface is alterable between a first shape that provides distant vision and a second shape that provides intermediate vision. In some embodiments, the diffractive optical surface is alterable to a third shape that provides near vision.

### BRIEF DESCRIPTION OF THE DRAWINGS

FIG. 1 is a cross sectional view of the human eye, with the lens in the unaccommodated state.

FIG. 2 is a cross sectional view of the human eye, with the lens in the accommodated state.

FIG. 3 schematically illustrates a cross sectional view of an embodiment of an intraocular lens implant having a diffractive optical surface.

FIG. 4 schematically illustrates a partial cross sectional view of the intraocular lens implant of FIG. 3.

FIG. 5 schematically illustrates a perspective view of an intraocular lens implant in an unaccommodated state.

FIG. 6 schematically illustrates a perspective view of the intraocular lens implant of FIG. 5 in an accommodated state.

FIG. 7 schematically illustrates a cross sectional view of an intraocular lens implant coupled with the ciliary muscle of an eye in an unaccommodated state.

FIG. 8 schematically illustrates a cross sectional view of the intraocular lens implant of FIG. 7 coupled with the ciliary muscle of an eye in an accommodated state.

FIG. 9 schematically illustrates a cross sectional view of an intraocular lens implant comprising two implants, one of which is in an unaccommodated state.

FIG. 10 schematically illustrates a cross sectional view of the intraocular lens implant of FIG. 9 with one of the implants in an accommodated state.

### DETAILED DESCRIPTION OF THE PREFERRED EMBODIMENTS

Many eye surgeries, such as cataract removals, involve the implantation of artificial lenses. Typically, artificial lenses have a fixed focal length or, in the case of bifocal or multifocal lenses, have several different fixed focal lengths. However, such fixed focal-length lenses lack the ability of the natural lens to dynamically change the optical power of the eye. Certain embodiments disclosed herein overcome this limitation, and additionally provide other advantages such as those described below.

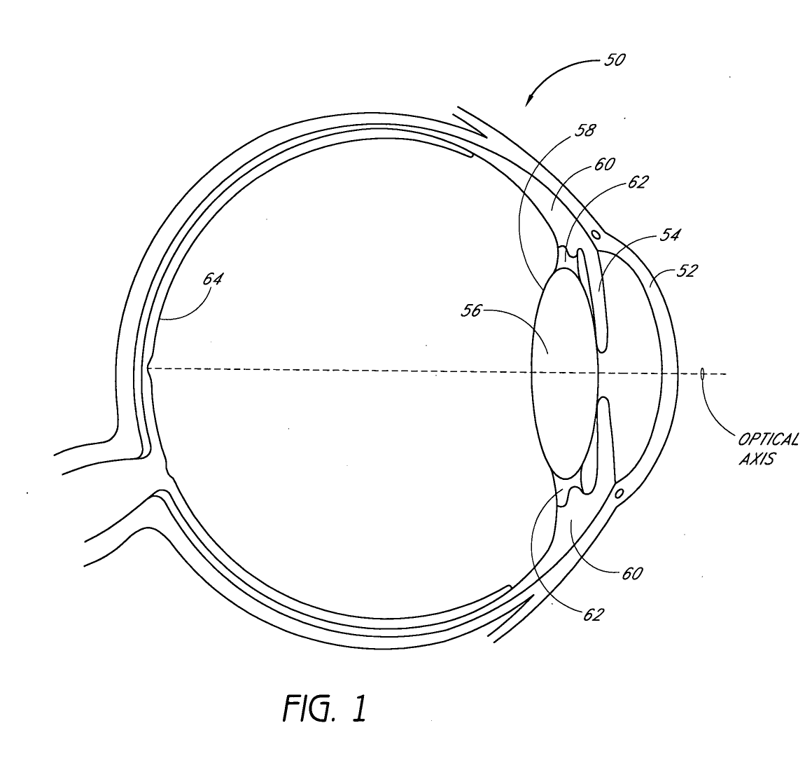

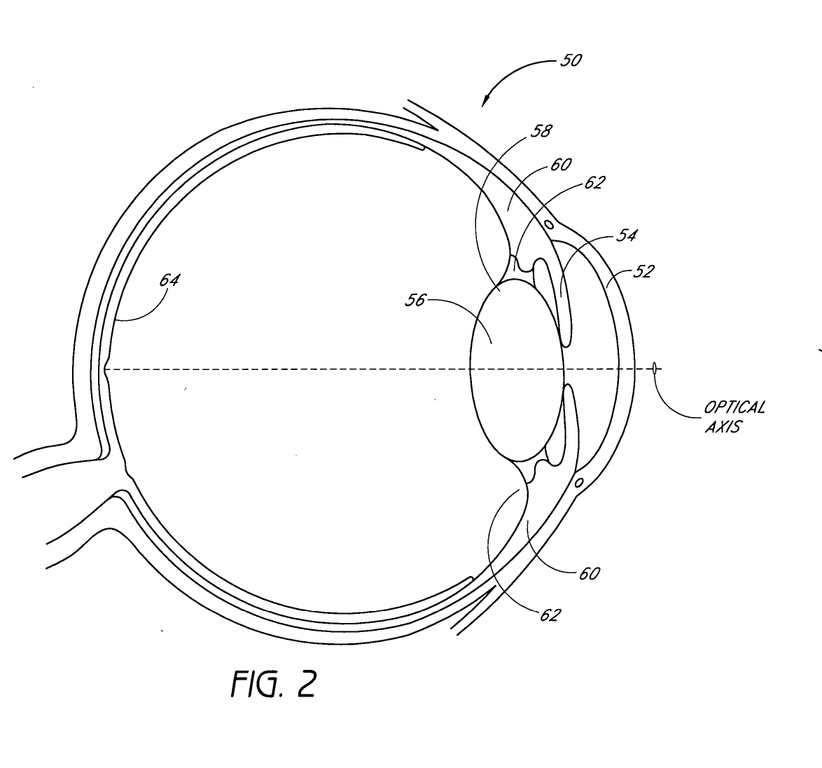

FIGS. 1 and 2 illustrate the human eye 50 in section. Of particular relevance to the present disclosure are the cornea 52, the iris 54 and the lens 56, which is situated within the elastic, membranous capsular bag or lens capsule 58. The capsular bag 58 is surrounded by and suspended within the ciliary muscle 60 by ligament-like structures called zonules 62.

As light enters the anterior portion of the eye 50, the cornea 52 and the lens 56 cooperate to focus the incoming light and form an image on the retina 64 at the posterior of the eye, thus facilitating vision. In the process known as accommodation, the shape of the lens 56 is altered (and its refractive properties thereby adjusted) to allow the eye 50 to focus on objects at varying distances. A typical healthy eye has sufficient accommodation to enable focused vision of objects ranging in distance from infinity (e.g., over about 20 feet from the eye) to very near (e.g., closer than about 10 inches).

The lens 56 has a natural elasticity, and in its relaxed state assumes a shape that in cross-section resembles a football. Accommodation occurs when the ciliary muscle 60 moves the lens from its relaxed or “unaccommodated” state (shown in FIG. 1) to a contracted or “accommodated” state (shown in FIG. 2). Movement of the ciliary muscle 60 to the relaxed/unaccommodated state increases tension in the zonules 62 and capsular bag 58, which in turn causes the lens 56 to take on a thinner (as measured along the optical axis) or taller shape, as shown in FIG. 1. In contrast, when the ciliary muscle 60 is in the contracted/accommodated state, tension in the zonules 62 and capsular bag 58 is decreased and the lens 56 takes on the fatter or shorter shape shown in FIG. 2. When the ciliary muscles 60 contract and the capsular bag 58 and zonules 62 slacken, some degree of tension is maintained in the capsular bag 58 and zonules 62.

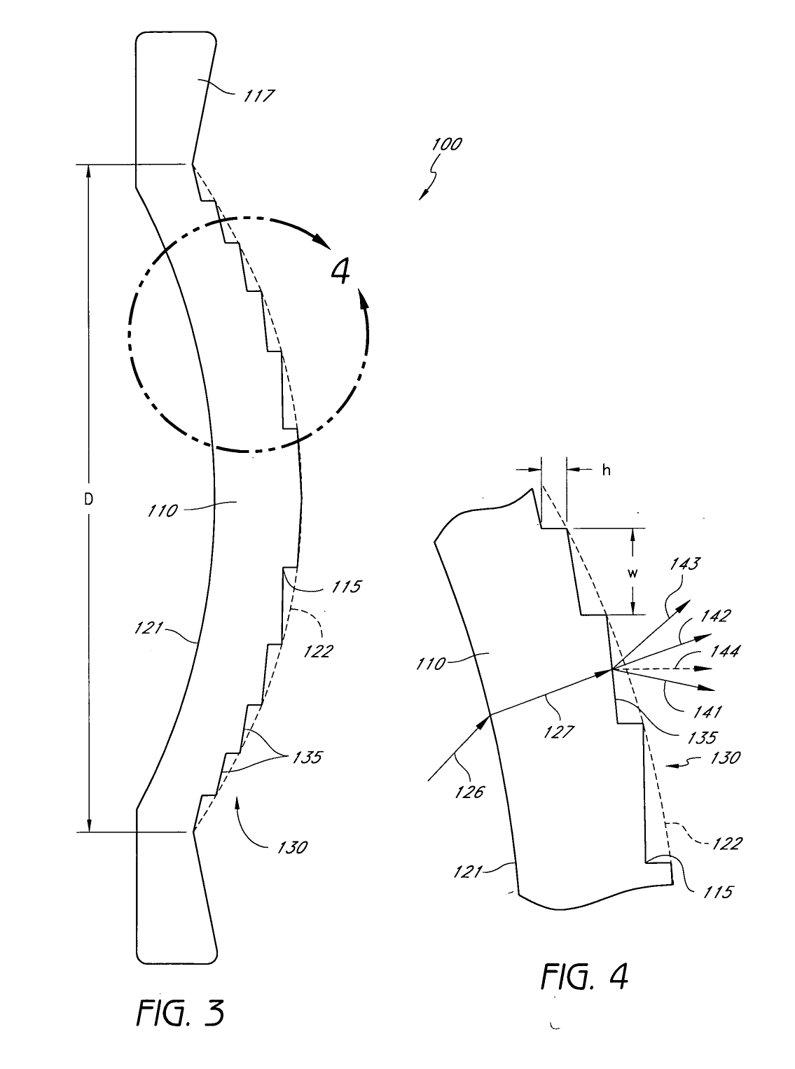

FIG. 3 schematically illustrates an embodiment of an intraocular lens implant 100, shown in cross section. In certain embodiments, the implant 100 comprises a lens body 110 sized and shaped for placement in an anterior portion of the eye 50, such as in the capsular bag 58. In some embodiments, the lens body 110 comprises a diffractive optical surface 115. The diffractive optical surface 115 can have diffractive properties which produce an interference pattern. In some embodiments, the lens body 110 is sufficiently flexible to change the shape of the diffractive optical surface 115 in response to action of the ciliary muscle 60 so that the interference pattern is modified. In further embodiments, accommodation is achieved by modification of the interference pattern. In some embodiments, the implant 100 comprises one or more haptics 117 configured to couple the lens body 110 with the eye 50.

In preferred embodiments, the lens body 110 is sufficiently compliant to change shape when the ciliary muscle 60 changes state for accommodation. In various embodiments, the lens body 110 comprises PMMA, silicone, soft silicone, polyhema, polyamide, polyimide, acrylic (hydrophilic or hydrophobic), or a shape memory material, or any suitable combination thereof. Other materials are also possible.

In certain embodiments, the implant 100 is sized and shaped for placement in an anterior portion of the eye 50. In some embodiments, the implant 100 is positioned in the capsular bag 58. In other embodiments, the implant 100 is positioned in the vitreous. In still further embodiments, the implant 100 is positioned in other areas of the anterior chamber of the eye 50, such as the sulcus or the iris plane.

With continued reference to FIG. 3, in various embodiments, a width (or in some embodiments, a diameter) D of the lens body 110 is between about 4 millimeters and about 8 millimeters, between about 5 millimeters and about 7 millimeters, or between about 5.5 millimeters and about 6.5 millimeters. In other embodiments, the width D is no more than about 6 millimeters, no more than about 7 millimeters, or no more than about 8 millimeters. In still other embodiments, the width D is no less than about 4 millimeters, no less than about 5 millimeters, or no less than about 6 millimeters. In preferred embodiments, the width D is about 6 millimeters.

In certain embodiments, the lens body 110 is shaped as a refractive lens that comprises one or more diffractive optical surfaces 115. For example, in the illustrated embodiment, the lens body 110 is generally shaped as a convex-concave lens, having a first surface 121 and a second surface 122, shown in phantom, each of which is substantially spherical. The lens body 110 can be shaped in any suitable configuration, including, without limitation, plano-convex, biconvex, or meniscus. The first and/or second surfaces 121, 122, also can be shaped in any suitable configuration, including, without limitation, aspheric configurations such as substantially planar, substantially spherical, substantially parabolic, or substantially hyperbolic. In many embodiments, the lens body 110 has refractive power due to the curvature of the first and second surfaces 121, 122.

In certain embodiments, the diffractive optical surface 115 follows a general contour or curvature of a substantially smooth base surface. In the illustrated embodiment, the base surface comprises the second surface 122. In many embodiments, the diffractive optical surface 115 further comprises a phase grating 130 that deviates from the contour or curvature of the base surface. As used herein, the term “grating” is a broad term used in its ordinary sense, and includes, without limitation, any feature of an optical element configured to produce an interference pattern. In some embodiments, the grating 130 includes an array, series, or pattern of grating regions 135, such as, for example, blaze zones, echelettes, or grooves. In some embodiments, the grating regions 135 are regularly spaced or periodic. The grating regions 135 can be formed in any suitable manner, such as, for example, by cutting or etching a blaze shape into the base surface (e.g., the second surface 122). In other embodiments, a layer, film, or coating is formed over the base surface (e.g., the second surface 122) to produce grating regions 135 that are raised with respect to the base surface. In still further embodiments, the lens body 110 is molded to include the grating regions 135. In some embodiments, the grating regions 135 comprise a series of concentric, step-like structures.

In various embodiments, the lens body 110 comprises a single diffractive optical surface 115. In other embodiments, the lens body 110 comprises a plurality of diffractive optical surfaces 115. One or more diffractive optical surfaces 115 can follow the general contours of the first and/or second surfaces 121, 122.

In some embodiments, the implant 100 comprises one or more haptics 117 configured to couple the lens body 110 with the eye 50. In preferred embodiments, the one or more haptics 117 are configured to couple with the ciliary muscle 60. In some embodiments, the haptics 117 extend outward from a periphery of the lens body 110, and can extend a sufficient distance from the lens body 110 to contact an edge of the capsular bag 58, the zonules 62, and/or the ciliary muscle 60. In certain embodiments, the haptics 117 are adhered or otherwise attached to the ciliary muscle 60 or the zonules 62 such that they move in response to contraction and/or relaxation of the ciliary muscle 60. In some embodiments, the haptics 117 are configured to abut the inner surface of the capsular bag 58 along some or all of a perimeter thereof, preferably near the zonules 62.

With reference to FIG. 4, in certain embodiments, light enters the lens body 110 through the first surface 121, as indicated by the arrow 126. The light propagates through the lens body 110, as indicated by the arrow 127, and exits through the diffractive optical surface 115. In certain embodiments, a periodic array of grating regions 135 scatters the exiting light, resulting in constructive and destructive interference of the light. Whether constructive or destructive interference occurs at an image plane of the lens body 110 depends on the difference in optical path length between separate grating regions 135, which is a function of the angles at which the light exits the grating regions 135 and the wavelength of the light.

In certain embodiments, the interference pattern created by the diffractive optical surface 115 comprises one or more diffraction orders. Constructive interference at a given point can result when portions of light from different grating regions 135 are in phase. Additionally, portions of light exiting different grating regions 135 that are phase shifted by a full wavelength, or by any number of full wavelengths, will constructively interfere. For example, in some embodiments, a zero diffraction order corresponds with an area where there is zero phase shift between portions of light coming from adjacent grating regions 135, a first diffraction order corresponds with an area where there is a one-wavelength phase shift, a second diffraction order corresponds with an area where there is a two-wavelength phase shift, and so on.

As illustrated in FIG. 4, in certain embodiments, each grating region 135 has a width w and a height h. In some embodiments, the width w of each grating region 135 is substantially the same. In further embodiments, the height h of each grating region 135 is substantially the same. Accordingly, in some embodiments, the diffraction grating 130 is periodic, and comprises a plurality of regularly spaced grating regions 135.

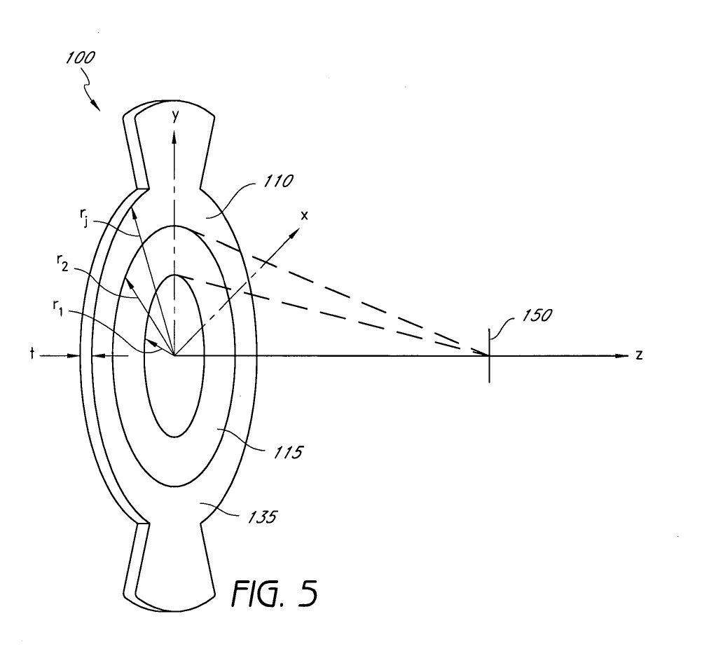

The period of the grating 130, which in some embodiments is equal to the width w of the grating regions 135, can affect the focal length or optical power of a given diffraction order. For example, the period of the grating 130 can affect the optical path length between different grating regions 135 and a given point. A difference in optical path length can result in a difference in phase between portions of light exiting the grating regions 135. As a result, a focal plane at which light constructively interferes (see, e.g., FIG. 5), and at which a diffractive image can be created, can move closer to or further from the lens body 110 as the period of the grating 130 changes. Thus, in certain embodiments, changing the width w of the grating regions 135 can change the distance of the focal plane from the lens body 110.

In certain embodiments, the height h of the grating regions 135 can affect the proportion of light that is directed to a given diffraction order. In some embodiments, light is channeled solely to the diffraction orders, and the percentage of total light exiting the lens body 110 that is channeled to a given order is referred to herein as the diffraction efficiency of this order. In the embodiment illustrated in FIG. 4, the arrows 141, 142, and 143 illustrate a geometrical model of three diffraction orders into which light of a given wavelength can be channeled: arrow 141 represents the −1 diffraction order; arrow 142 represents the 0 diffraction order; and arrow 143 represents the +1 diffraction order. Arrow 144 illustrates the blaze ray, which is the direction at which light is refracted out of the lens body 110 at the grating region 135. In certain embodiments, it is possible to achieve a diffraction efficiency of approximately 100% for a given diffraction order when the blaze ray 144 and the arrow representing the diffraction order coincide. Accordingly, it is possible to vary the percentage of light directed to a given diffraction order by altering the height h of the grating region 135.

FIG. 5 schematically illustrates a perspective view of an embodiment of the intraocular lens implant 100. A center of the lens body 110 is shown at the origin of an xyz coordinate system for illustrative purposes. In certain embodiments, an optical axis of the lens body 110 extends through the center of the lens body 110. In the illustrated embodiment, the optical axis coincides with the z axis. In some embodiments, the lens body 110 has a thickness t, as measured in a direction parallel to the z axis.

In certain embodiments, the diffractive optical surface 115 comprises a series of concentric grating regions 135. In the illustrated embodiment, the grating regions 135 are circular, as is the periphery of the lens body 110. In various other embodiments, the grating regions 135 and/or lens body 110 can define other shapes, such as ovals, ellipses, or polygons, for example. The grating regions 135 also can be arranged in patterns other than concentric. In the illustrated embodiment, each circular grating region 135 has a radius of a different length, as indicated by the arrows r₁, r₂, and rⱼ. In certain embodiments, the diffractive optical surface 115 channels light into one or more diffractive orders. A single diffractive order is represented in FIG. 5 by an image plane 150.

In certain embodiments, the spacing of the grating regions 135 is defined according to the following equation:
rⱼ²+f²=(f+jmλ) (1)
where m is the given diffractive order, f is the focal length of the given diffractive order, λ is the wavelength of light, and rⱼ is the radius of a given grating region 135, where j is an positive integer.

In simple paraxial form, equation (1) can be reduced as follows: rⱼ²=jmλf. Accordingly, the focal length of the m^{th} diffraction order can be approximated by the equation:

$${f}_{m} = \frac{{r}_{j}^{2}}{jm λ} \qquad ( 2 )$$

Additionally, a paraxial approximation of the height h of the grating regions 135 that will produce a diffraction efficiency of approximately 100% for the m^{th} diffraction order in certain embodiments is as follows:

$${h}_{m} = \frac{m λ}{( n - {n}^{′} )} \qquad ( 3 )$$

where n is the refractive index of the material of the lens body 110 and n′ is the refractive index of the material surrounding the lens body 110. In certain embodiments, the implant 100 is within the capsular bag 58 and the lens body 110 is surrounded by an aqueous material having an index of refraction of about 1.336.

In certain embodiments, the parameters rⱼ and hₘ can be selected to produce a lens body 110 of a given focal length fₘ. For example, the focal length fₘ can be determined by the IOL power calculation. Advantageously, in such embodiments, the focal length fₘ is independent of the thickness t of the lens body 110. Accordingly, in some embodiments, the lens body 110 can be relatively thin, which can permit the diffractive optical surface 115 to readily change shape in response to movement of the ciliary muscle 60.

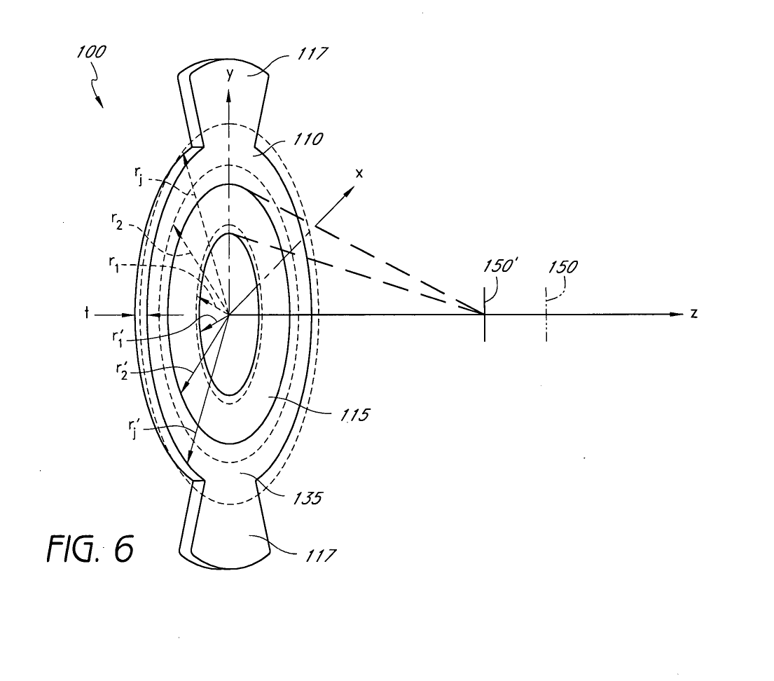

FIG. 6 schematically illustrates the implant 100 in a changed configuration in response to movement of the ciliary muscle 60. In certain embodiments, movement of the ciliary muscle 60 causes the diffractive optical surface 115 to change shape. In many embodiments, the diffractive optical surface 115 is elastically deformed from one shape to another. In some embodiments, a curvature of the diffractive optical surface 115 changes as the ciliary muscle 60 moves. For example, in some embodiments, the optical surface 115 bends, bows, or arcs in response to the muscle movement, and in other embodiments, the optical surface 115 stretches, flattens, or compresses, in response to movement of the ciliary muscle 60.

In certain embodiments, the lens body 110 is in an unaccommodated state when the shape of the diffractive optical surface 115 is unchanged and is in an accommodated state when the shape of the diffractive optical surface is changed. In some embodiments, when the ciliary muscle 60 is in a relaxed condition, the lens body 110 and diffractive optical surface 115 generally assume their natural shape. When the ciliary muscle 60 contracts for accommodation, it applies force to the haptics 117 and changes the shape of the lens body 110 and the diffractive optical surface 115. In some embodiments, the base surface (e.g., the second surface 122) of the diffractive optical surface 115 is more highly curved when the lens body 110 is in the accommodated state than is the base surface when the lens body 110 is in the unaccommodated state.

In other embodiments, the lens body 110 is in a natural or relatively unstressed state when the ciliary muscle 60 is contracted for accommodation. In certain of such embodiments, as the ciliary muscle 60 relaxes, it pulls on the haptics 117 to change the shape of the lens body 110 and the diffractive optical surface 1115. In some embodiments, the base surface of the diffractive optical surface 115 becomes less rounded as the ciliary muscle 60 relaxes.

In some embodiments, the change in curvature of the base surface of the diffractive optical surface 115 is substantially uniform along multiple cross sections of the lens body 110. For example, in some embodiments, when the shape of the diffractive optical surface 115 is unchanged, a cross section of the lens body 110 along the xz plane, as defined in FIG. 6, reveals a curvature of the base surface that is substantially the same as the curvature of the base surface along the yz plane. As the shape of the diffractive optical surface 115 changes, the changing curvature of the base surface along the xz plane and that of the base surface along the yz plane remain substantially the same as each other. In further embodiments, the curvature of the base surface along multiple planes that (i) are perpendicular to the xy plane and (ii) extend through the optical axis (i.e., the z axis) are substantially the same throughout a change in shape of the diffractive optical surface 115.

In certain embodiments, the manner in which the optical surface 115 changes shape is affected by the material and/or the configuration of the lens body 110. In certain embodiments, the flexibility at a central region of the lens body 110 is different than the flexibility at an outer region of the lens body 110. For example, in some embodiments, either the stiffness or the compliance of the material of the lens body 110 increases toward the center of the lens body 110. In further embodiments, the lens body 110 comprises a first material at an outer region and a second material at a central region, and the first material can be more or less compliant than the second material. In still further embodiments, the lens body 110 comprises a plurality of materials having different flexibilities.

In some embodiments, the thickness t varies between a center of the lens body 110 and the periphery thereof. The thickness t can increase or decrease toward the center of the lens body 110. In other embodiments, the thickness t is substantially constant. In many embodiments, regions of the lens body 110 that are relatively more compliant and/or are thinner can be reshaped to a larger degree than relatively stiffer and/or thicker portions of the lens body 110.

In some embodiments, the manner in which the lens body 110 is coupled with the ciliary muscle 60 affects the manner in which the lens body 110 changes shape. In some embodiments, a plurality of haptics 117 extend from the periphery of the lens body 110. The haptics 117 can be pulled in different directions along a common plane such that the curvature of the lens body 110 changes in a substantially uniform manner. In some instances, a greater uniformity in a change of curvature can result from a relatively larger number of haptics 117. In other embodiments, the periphery of the lens body 110 is coupled with the ciliary muscle 60 via an assembly or mechanism comprising a spring coil member and haptics. Embodiments of such a device are disclosed in U.S. patent application Ser. No. 10/016,705, filed Dec. 10, 2001, titled ACCOMMODATING INTRAOCULAR LENS, the entire contents of which are hereby incorporated by reference herein and made a part of this specification. In certain embodiments, such a device can constrict the lens body 110 about its peripheral edge to effect a relatively uniform change in the shape of the lens body 110 as the ciliary muscle 60 relaxes and contracts. Other systems and methods are also possible for coupling the lens body 110 with the ciliary muscle 60.

As illustrated in FIG. 6, in certain embodiments, the distance between different grating regions 135 and the optical axis of the lens body 110 changes as the diffractive optical surface 115 changes shape. In the illustrated embodiment, the radii of the circular grating regions 135 are reduced as compared with those in FIG. 5. This is indicated by the grating regions 135 shown in phantom and by the arrows r₁′, r₂′, and rⱼ′, which are relatively shorter than the arrows r₁, r₂, and rⱼ. In some embodiments, the lens body 110 is compressed or stretched such that the radii of the grating regions 135 are reduced or expanded, respectively, while the curvature of the diffractive optical surface 115 does not change significantly. In other embodiments, the curvature of the diffractive optical surface 115 becomes more or less bowed such that the grating regions 135 move closer to or further from the optical axis of the lens body 110. In some embodiments, the grating regions 135 become more or less closely spaced to each other, as measured in a direction perpendicular to the optical axis.

In certain embodiments, the radii of the grating regions 135 are reduced proportionally to the amount that the curvature of the base surface of the diffractive optical surface 115 changes, which can shift the image plane 150 toward the diffractive optical surface 115. In some embodiments, the diameter of the lens body 110 is between about 4 millimeters and about 8 millimeters. In certain of such embodiments, contraction of the ciliary muscle 60 urges the periphery of the lens body 110 towards the center of the lens body 110 by about 0.25 millimeters, which produces a relatively small change in the curvature of the base surface of the diffractive optical surface 115. In some embodiments, this change in curvature can vary the orientation of the grating regions 135. For example, each grating region 135 can be generally planar in an unchanged state, and can be angled to a slightly frustoconical shape in a changed state. However, in the small range of change effected by movement of the ciliary muscle 60, the small angle approximation of α≈sin(α) can apply. Accordingly, the changed diffractive optical surface 115 can still produce distinct diffractive orders, and the grating regions 135 can still follow equations (1), (2), and (3). As a result, according to equation (2), the focal length fₘ of a given diffraction order will be smaller for the changed diffractive optical surface 115, since the radii r₁′, r₂′, and rⱼ′ are smaller than the radii r₁, r₂, and rⱼ (shown in phantom).

Accordingly, in certain advantageous embodiments, changing the shape of the diffractive optical surface 115 produces a gain in optical power, thus allowing the implant 100 to be used for accommodation. As illustrated in FIG. 6, the image plane 150′ of a given diffractive order is closer to the diffractive optical surface 115 than the image plane 150 (shown in phantom). The focus of the implant 100 can thus be shifted from distant vision to near vision, or vice versa, by changing the shape of the diffractive optical surface 115. Advantageously, in preferred embodiments, the implant 100 further allows a range of intermediate vision between distant and near vision, and in further embodiments, the range of intermediate vision is continuous.

In certain embodiments, the height h and width w of the grating regions 135 are such that approximately 100% of the optical output of the diffractive optical surface 115 is channeled to a single diffraction order, which can be designated as the “design” diffraction order. Accordingly, the diffraction efficiency of the design diffraction order is approximately 100%. As described above, the distance of the image position of the design diffraction order from the diffractive optical surface 115, i.e., the focal length of the diffractive optical surface 115, can be altered by changing the shape of the diffractive optical surface 115. However, in certain embodiments, changing the shape of the diffractive optical surface 115 can cause minor deformations of the height h and width w and, as noted above, can also change the relative orientation of the grating regions 135. In some embodiments, these changes can channel some of the optical output to other diffraction orders, thereby reducing the diffraction efficiency of the design diffraction order.

In many instances, a small reduction in contrast is acceptable for near vision. Accordingly, in preferred embodiments, distant vision is produced by the diffractive optical surface 115 when its shape is unchanged, and near vision is produced when its shape is changed. In some embodiments, the diffractive optical surface 115 channels about 100% of the light entering the lens body 110 to the design diffraction order when the shape of the diffractive optical surface 115 is unchanged.

In preferred embodiments, a relatively large percentage of the optical output of the diffractive optical surface 115 is directed to the design diffraction order for distant, intermediate, and near vision. In various embodiments, at least about 50%, at least about 60%, at least about 70%, at least about 75%, at least about 80%, at least about 85%, at least about 90%, or at least about 95% of the optical output of the diffractive optical surface 115 is directed to the design diffraction order.

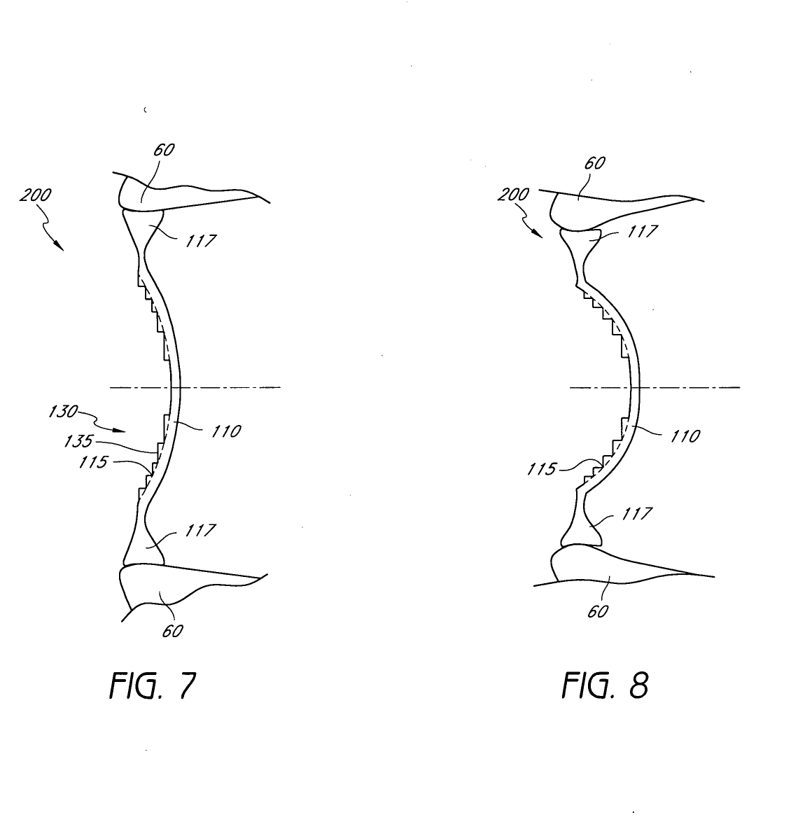

FIGS. 7 and 8 schematically illustrate an embodiment of an intraocular lens implant 200 in an unaccommodated state and in an accommodated state, respectively. The implant 200 is similar to the implant 100 in many respects. Accordingly, like features of the implants 100, 200 are identified with like numerals. In certain embodiments, the implant 200 comprises a lens body 110, a diffractive optical surface 115, and a plurality of haptics 117. The optical surface 115 can comprise a grating 130 having a plurality of grating regions 135.

In certain embodiments, a method comprises providing the implant 200. The method further comprises implanting the implant 200 in the eye 50. In certain embodiments, the implant 200 is coupled with the ciliary muscle 60. In some embodiments, the curvature of the diffractive optical surface 115 changes in response to movement of the ciliary muscle 60.

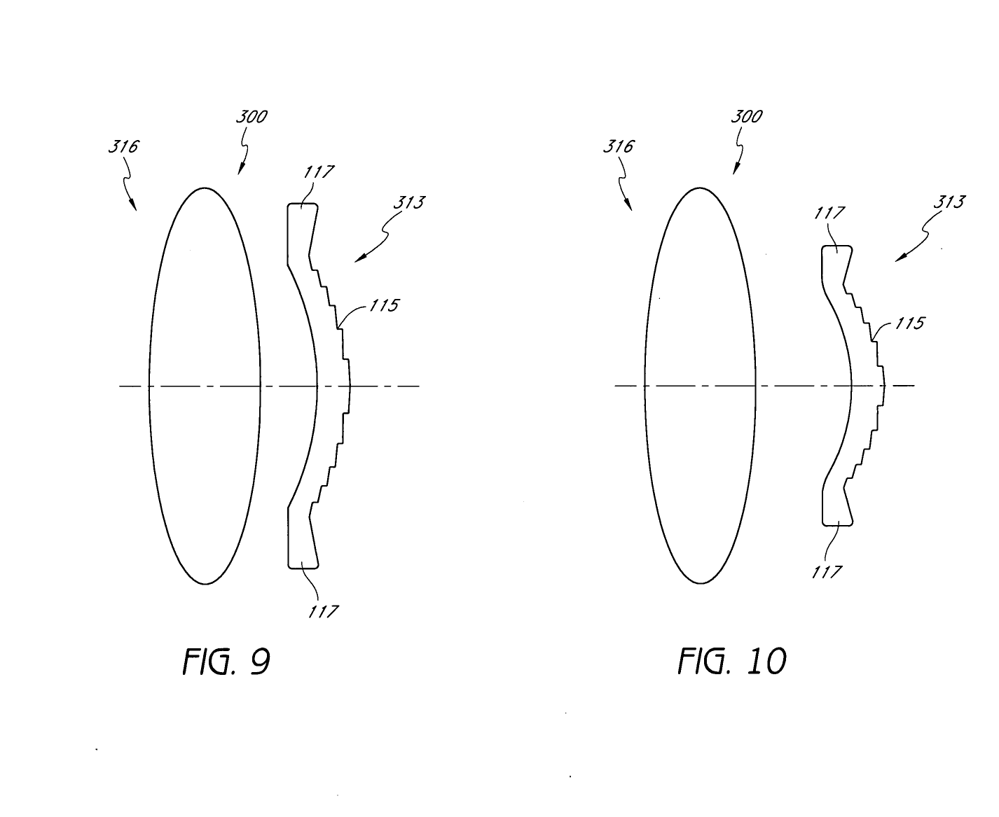

FIGS. 9 and 10 schematically illustrate an embodiment of an intraocular lens implant 300 in an unaccommodated state and in an accommodated state, respectively. In certain embodiments, the implant 300 comprises a first implant 313, such as the implants 100 and 200 described above, and a second implant 316. In some embodiments, the first implant 313 comprises a diffractive optical surface 115 configured to change shape. In further embodiments, the first implant 313 comprises one or more haptics 117 for coupling with the ciliary muscle 60. In some embodiments the second implant 316 is configured to change shape in response to action of the ciliary muscle 60, while in other embodiments, the second implant 316 is not configured to change shape. In various embodiments, the second implant 316 is anterior to or posterior to the first implant 313.

In some embodiments, the second implant 316 comprises one or more refractive optical surfaces. In some embodiments, the second implant 316 comprises a refractive lens. In some advantageous embodiments, the first and second implants 313, 316 are configured to move relative to one another when the eye accommodates. In certain of such embodiments, the first implant 313 does not significantly change shape when the eye 50 accommodates. Accordingly, in some embodiments, the diffraction efficiency of the design diffraction order of the first implant 313 can be near 100% for distant, intermediate, and near vision.

In some embodiments, the second implant 316 is a diffractive optic. In further advantageous embodiments, the second implant 316 is a multiphase diffractive optic, which can reduce the impact of chromatic aberration from the first implant 313. In further embodiments, two or more optics are combined with the first implant 313 in a multi-lens and/or multi-optic system.

Although the inventions presented herein have been disclosed in the context of certain preferred embodiments and examples, it will be understood by those skilled in the art that the inventions extend beyond the specifically disclosed embodiments to other alternative embodiments and/or uses of the inventions and obvious modifications and equivalents thereof. Thus, it is intended that the scope of the inventions herein disclosed should not be limited by the particular embodiments described above, but should be determined only by a fair reading of the claims that follow.

What is claimed is:

1. A method comprising:
providing an intraocular lens comprising a diffractive optical surface having diffractive properties which produce an interference pattern;
implanting said lens in an eye of a patient such that said diffractive optical surface changes shape in response to action of an ocular structure of the eye,
whereby said interference pattern is modified in response to said action of said ocular structure.

2. The method of claim 1, wherein said interference pattern comprises a diffractive image.

3. The method of claim 1, wherein at least about 80 percent of the optical output of said diffractive optical surface is in a single diffraction order.

4. The method of claim 1, wherein said ocular structure comprises the ciliary muscle of the eye.

5. The method of claim 1, wherein said diffractive optical surface comprises a grating comprising a plurality of grating regions.

6. The method of claim 5, wherein a distance between at least some of the plurality of grating regions in a direction perpendicular to an optical axis of the intraocular implant changes as the shape of said diffractive optical surface is changed.

7. The method of claim 1, further comprising implanting another intraocular lens having a refractive optical surface.

8. The method of claim 1, wherein the curvature of a base surface of said diffractive optical surface changes due to the change in shape of said diffractive optical surface.

9. The method of claim 1, further comprising coupling the periphery of said lens with the ciliary muscle of the eye.

10. The method of claim 9, wherein one or more haptics are coupled with the ciliary muscle of the eye.

11. The method of claim 9, wherein a peripheral spring coil member is coupled with the ciliary muscle of the eye.

12. An intraocular implant comprising:
a lens body comprising a diffractive optical surface having diffractive properties which produce an interference pattern, said lens body being sized and shaped for placement in an anterior portion of a human eye, said lens body being sufficiently flexible to change the shape of said diffractive optical surface in response to ciliary muscle action so that said interference pattern is modified.

13. The intraocular implant of claim 12, wherein at least about 80 percent of the optical output of said diffractive optical surface is in a single diffraction order.

14. The intraocular implant of claim 12, wherein said implant is in an unaccomodated state when the shape of said diffractive optical surface is unchanged and is in an accommodated state when the shape of said diffractive optical surface is changed.

15. The intraocular implant of claim 12, wherein said interference pattern comprises one or more diffraction orders and wherein a distance, along an optical axis of said lens body, between (i) at least one of said one or more diffraction orders and (ii) said lens body changes as the shape of said diffractive optical surface is changed.

16. The intraocular implant of claim 12, wherein said diffractive optical surface comprises a grating comprising a plurality of grating regions.

17. The intraocular implant of claim 16, wherein a distance between one or more of the plurality of grating regions and an optical axis of said intraocular implant changes as the shape of said diffractive optical surface is changed.

18. The intraocular implant of claim 12, further comprising a second lens with a refractive optical surface.

19. The intraocular implant of claim 12, wherein the curvature of a base surface of said diffractive optical surface is changed when the shape of said diffractive optical surface is changed.

20. The intraocular implant of claim 19, wherein the curvature is substantially uniform along multiple cross sections of said lens body.

21. The intraocular implant of claim 12, wherein the flexibility at a central region of said lens body is different than the flexibility at an outer region of said lens body.

22. The intraocular implant of claim 21, wherein said lens body is thinner at said outer region thereof than at said central region thereof.

23. The intraocular implant of claim 21, wherein said lens body comprises a first material at said outer region thereof and a second material at said central region thereof, said first material being more compliant than said second material.

24. An intraocular implant comprising:
an optical element sized for insertion into a human eye, said optical element having a diffractive optical surface, said diffractive optical surface having an unaccommodated state in which said diffractive optical surface creates a first interference pattern and an accommodated state in which said diffractive optical surface creates a second interference pattern which differs from the first interference pattern, said optical element being sufficiently flexible to change from said unaccommodated state to said accommodated state in response to ciliary muscle action.

25. The intraocular implant of claim 24, wherein said first interference pattern comprises a first image position of a diffraction order and said second interference pattern comprises a second image position of said diffraction order, said first and second diffractive image positions being spaced from each other.

26. The intraocular implant of claim 24, wherein a base surface of said diffractive optical surface is more highly curved in said accommodated state than in said unaccommodated state.

27. The intraocular implant of claim 24, wherein said first and second interference patterns each comprises one or more diffraction orders, said one or more diffraction orders being spaced further from said optical element when said diffractive optical surface is in said unaccommodated state than when said optical element is in said accommodated state.

28. The intraocular implant of claim 24, wherein said diffractive optical element comprises a plurality of gratings having a uniform grating width.

29. An intraocular implant comprising:
an optical element sized for insertion into a human eye, said optical element having a diffractive optical surface, said diffractive optical surface being alterable between a first shape that provides distant vision and a second shape that provides intermediate vision.

30. The intraocular implant of claim 29, wherein said diffractive optical surface is alterable to a third shape that provides near vision.

31. The intraocular implant of claim 29, wherein said diffractive optical surface creates an interference pattern having one or more diffraction orders.

32. The intraocular implant of claim 31, wherein a single diffraction order provides said distant vision and said intermediate vision.
